(19) United States
(12) Patent Application Publication
(10) Pub. No.: US 2020/0323786 A1
(43) Pub. Date: Oct. 15, 2020

(54) GROWTH-FACTOR NANOCAPSULES WITH TUNABLE RELEASE CAPABILITY FOR BONE REGENERATION

(71) Applicants: The Regents of the University of California, Oakland, CA (US); THE UNIVERSITY OF SOUTHERN CALIFORNIA, Los Angeles, CA (US); TIANJIN UNIVERSITY, Tianjin (CN)

(72) Inventors: Yunfeng Lu, Culver City, CA (US); Jeffrey C. Wang, Sherman Oaks, CA (US); Haijun Tian, Shanghai (CN); Juanjuan Du, San Diego, CA (US); Jing Wen, Culver City, CA (US); Yang Liu, Los Angeles, CA (US); Xubo Yuan, Tianjin (CN)

(73) Assignees: THE REGENTS OF THE UNIVERSITY OF CALIFORNIA, Oakland, CA (US); THE UNIVERSITY OF SOUTHERN CALIFORNIA, Los Angeles, CA (US); TIANJIN UNIVERSITY, Tianjin, CA (CN)

(21) Appl. No.: 16/304,116

(22) PCT Filed: May 24, 2017
(86) PCT No.: PCT/US2017/034330

§ 371 Date: Nov. 21, 2018

(51) Int. Cl. A61K 9/51; A61K 38/18; B82Y 5/00; A61P 19/00

(52) U.S. Cl. CPC A61K 9/5138; A61K 38/1875; A61P 19/00; B82Y 5/00; A61K 9/5192

(57) ABSTRACT

Growth factors are of great potential in regenerative medicine. However, their clinical applications are largely limited in by short in vivo half-lives and a narrow therapeutic window. Thus, a robust controlled release system remains an unmet medical need for growth-factor-based therapies. A nanoscale controlled release system (degradable protein nanocapsule) is provided via in-situ polymerization on growth factor. The release rate can be finely tuned by engineering the surface polymer composition. Improved therapeutic outcomes are achieved with the growth factor nanocapsules, as illustrated in spinal cord fusion mediated by bone morphogenetic protein-2 (BMP-2) nanocapsules.

### REFERENCE TO RELATED APPLICATIONS

This application claims priority under Section 119(e) from U.S. Provisional Application Ser. No. 62/340,882, filed May 24, 2016, entitled “GROWTH-FACTOR NANOCAPSULES WITH TUNABLE RELEASE CAPABILITY FOR BONE REGENERATION” by Yunfeng Lu et al., the contents of which are incorporated herein by reference.

### TECHNICAL FIELD

The invention relates to nanocapsules and in particular, the encapsulation and controlled release of cargo such as proteins.

### BACKGROUND OF THE INVENTION

Growth factors play important roles in stimulating cell growth, regulating cell proliferation and differentiation, and controlling the formation of the extracellular matrix. Over the past decades, a number of researches and trials have been performed to evaluate the effectiveness of growth factors for tissue repair and regeneration [1], where maintaining suitable levels of growth factors in the target tissue is highly desired [2, 3]. Similar to most proteins, however, growth factors are mostly unstable and short-lived in vivo [4]. Since tissue regeneration or repair is usually a long-lasting process, developing strategies that can stably and persistently release the growth factors is crucial for the healing process [5].

To date, various approaches have been explored for growth factor delivery. Among them, hydrogel-based systems probably have received the most attention [6]. In these systems, growth factors are directly embedded within the hydrogel, often resulting in a burst release of the growth factors upon swelling of the hydrogels [5, 7-9]. To control the release profile, additional treatments have been introduced, such as crosslinking the hydrogels [10-13] and conjugating growth factors onto the hydrogels [14]. A major concern of these strategies is that the crosslinking and conjugation reactions may compromise the activity of the growth factors [15]. Besides hydrogels, growth factors have been embedded within other polymer matrices (e.g., poly(lactide-co-glycolic acid) and poly(c-caprolactone)) by layer-by-layer assembly [16], electrospinning [17], biphasic assembly or high-pressure CO₂ fabrication [18, 19]. These strategies enable the formation of growth-factor composites in the forms of films, scaffolds, or microparticles. Tuning degradation kinetics of the polymer matrix enables controlled release of the growth factors [14, 20-23]. However, the synthesis of such composites often requires harsh chemical processes involving intense mixing and/or use of organic solvents, which can easily denature the growth factors.

A specific growth factor, bone morphogenetic protein-2 (BMP-2), is commonly used to enhance bone regeneration in association with orthopedic surgeries [28]. Since its approval for clinical use by the U.S. Food and Drug Administration (FDA) in 2002, BMP-2 has achieved wide-spread use because its osteogenic effect allows it to substitute bone autograft or allograft [29]. The challenge in using BMP-2 for bone regeneration is the inherent short half-life the protein exhibits in vivo, as well as the short local residence time and high cost. In addition, the most prominent and dangerous side effect of BMP-2 is the associated inflammatory reaction [30]. Although a local inflammatory reaction is required to initiate the subsequent process of tissue regeneration, excessive inflammation may lead to untoward side effects [31, 32]. Furthermore, overdosed BMP-2 induces adipogenesis in addition to osteogenesis [33], leading to low bone quality. Therefore, maintaining the concentration of BMP-2 within a narrow therapeutic widow is critically important in order to achieve an optimal therapeutic outcome. Higher concentrations lead to side effects such as inflammation reactions whereas lower concentrations do not have a therapeutic effect. Moreover, the time span in which BMP-2 level is maintained in the therapeutic window is more important for the therapeutic outcome. To date, multiple strategies for sustained release of BMP-2 have been explored [34-36]. A delivering system with effective osteogenisity and reduced side effects, however, has yet to be demonstrated in the current art.

Thus, there is a need in the art for improved methods and compositions for delivering polypeptides such as growth factors to in vivo targets. This includes a need for methods and compositions for administering polypeptides such as BMP-2 for bone regeneration with effective osteogenisity and reduced side effects. The invention disclosed herein meets these needs via a novel protein delivery system comprising of polymer nanocapsules that encapsulate proteins within degradable polymer shells that have tunable release capability. As discussed below, this protein delivery system allows for the controlled and sustained release of a protein in vivo.

### SUMMARY OF THE INVENTION

The invention disclosed herein provides a nanoscale controlled-release system designed to control the sustained release of a protein cargo (e.g. a growth factor such as bone morphogenetic protein-2) in vivo in a manner that preserves the bioactivity of that cargo as well as methods for using this system. Embodiments of the invention include polymer nanocapsules whose rate of degradation in vivo can be precisely controlled in order to stably and persistently release protein cargo within a defined therapeutic window. The working examples presented below confirm that the constellation of elements in this new system can mitigate side effects observed in conventional regimens used to delivery polypeptide therapeutics and further provide improved therapeutic outcomes.

As demonstrated in illustrative experiments below that were designed to facilitate bone regeneration, the sustained release and delivery of bone morphogenetic protein-2 (BMP-2) from the nanocapsules disclosed herein successfully mediated bone development, leading to bone regeneration with improved bone quality. Importantly, the sustained release and delivery of BMP-2 reduced the side effects associated with the excessive use of native BMP-2 in traditional spinal cord fusion surgery, thereby providing a safe and more effective BMP-2 therapy for bone regeneration. By replacing BMP-2 with different protein cargos, this controlled-release system may be further extended to other therapeutic proteins in a variety of clinical applications.

The invention disclosed herein has a number of embodiments. One embodiment is a composition of matter that includes a polymer nanocapsule comprising a protein cargo and a degradable polymer shell encapsulating the protein cargo. The polymer shell is typically cationic and is formed from one or more different monomers and at least one crosslinker having a bond that degrades in an alkaline environment. The degradation rate of the polymer shell is controlled by the selected crosslinker and/or by changing the ratio of the one or more different monomers. Typically, the composition is provided as a population of polymer nanocapsules having varying amounts of crosslinkers and/or ratios of the one or more different monomers, thereby providing a variable and sustained release of the protein cargo in a basic environment.

Embodiments of the invention include methods for making and using the polymer nanocapsules disclosed herein. For example, one embodiments is a methods for making embodiments of the invention by selecting a core cargo molecule for encapsulation, as well as a plurality of shell monomers and/or cross-linkers having moieties that degrade at a pH of 7.4 or above. In these embodiments, amounts of crosslinkers and/or monomers used to make the thin polymer shell can be varied so as to form a population of nanocapsules having varying amounts of crosslinkers and/or different amounts of monomers disposed therein. In such embodiments, the amounts of crosslinkers and/or monomers varied to form a population of nanocapsules that are designed to variably degrade in alkaline environments such as sites of bone healing in vivo.

In a working embodiment of the invention disclosed below, a method for stimulating bone regeneration is provided. The method comprises delivering a polymer nanocapsule to bone tissue and degrading the polymer shell such that a bone morphogenetic protein-2 (BMP-2) growth factor is released at the bone tissue and stimulates osteoinduction. The polymer nanocapsule comprises a bone morphogenetic protein-2 (BMP-2) growth factor and a degradable polymer shell encapsulating the protein cargo. The polymer shell comprises polymerized N-(3-aminopropyl) methacrylamide (APm) and acrylamide (AAm) monomers and glycerol dimethacrylate (GDMA) crosslinkers.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

Referring now to the drawings in which like reference numbers represent corresponding parts throughout:

FIG. 1. (FIG. 1A) TEM image of negatively stained bovine serum albumin nanocapsules (nBSA) (Inset: a TEM image of the positively stained nanocapsules). (FIG. 1B) Agarose gel electrophoresis of nBSA synthesized using acrylamide (AAm) and N-(3-aminopropyl) methacrylamide (APm) as monomer before and after the treatment in basic condition for 6 days. Non-degradable crosslinker bisacrylamide (BIS) or degradable crosslinker glycerol dimethacrylate (GDMA) was used, which are denoted as nBSA(BIS) and nBSA(GDMA), respectively. (FIG. 1C) Agarose gel electrophoresis of the nanocapsules synthesized using AAm and 2-(dimethylamino)ethyl methacrylate (DMA) as the monomers before and after treatment in basic condition for 2 days. Non-degradable crosslinker BIS or degradable crosslinker GDMA was used, which are denoted as nBSA(BIS) or nBSA(GDMA), respectively. (FIG. 1D) Agarose gel electrophoresis of the nanocapsules synthesized with various molar ratios of APm and DMA as the monomers and GDMA as the crosslinker over a 6-day incubation in basic environment. (FIG. 1E) Release rate of the BSA cargo from nBSA made with different APm/DMA ratios and the GDMA crosslinker. The release of BSA from the degradable nanocapsules was quantified for the gel of FIG. 1D and the data is summarized in FIG. 1E. FIGS. 1D and 1E are more comprehensive studies of the release kinetics with multiple time points and polymer composition. They offer more quantitative information than FIG. 1B. * The half-life of nBSA with an APm/DMA ratio of 1 is based on the estimation by fitting the released BSA concentration into the same model as the other three groups.

FIG. 2. Characterization and in-vitro test of release kinetics and osteogenic property of BMP-2 nanocapsule: (FIG. 2A) Represented TEM image of negatively stained nBMP-2; (FIG. 2B) Hydrodynamic size distribution of nBMP-2 nanocapsules determined by dynamic light scattering; (FIG. 2C) ELISA test showing degradation of nBMP-2; (FIG. 2D) ALP activity in C3H10T1/2 cells after treated with native BMP-2 or nBMP-2 before and after a 3-day incubation of BMP-2 and nBMP-2 under pH 8.5. ALP activity is determined by integrated optical density (TOD) in C3H10T1/2 cells stained with ALP staining kit.

FIG. 3. In-vivo test of nBMP-2: (FIG. 3A) Fusion score of different animal groups using a rat spinal fusion model at 8 weeks, nMBP-2 concentration is equivalent to 1.5 μg BMP-2; (FIG. 3B) Representative CT images of BMP-2 and nBMP-2 treated rat spines at 8 weeks, showing nBMP-2 group has a relatively smoother surface, indicating better bone quality; (FIG. 3C) Quantified bone volume data confirms that nBMP-2 group has a higher relative bone volume (BV/TV), *** p<0.001; (FIG. 3D) Histology shows that the fusion mass of the BMP-2 group was occupied by large amount of adipose cells, while the nBMP-2 group has more trabecular bone inside. The analysis is done on rats after treatment of BMP-2 and nBMP-2 for 8 weeks. (FIG. 3E) Gross image of subcutaneous seroma in a rat treated with BMP-2 and nBMP-2 2 days after surgery. BMP-2 has the most significant seroma leakage due to the inflammatory effect; (FIG. 3F) Representative MR images and histology images of rat spinal cord and peripheral tissue 2 days after implanting with BMP-2, nBMP-2 or PBS containing collagen sponges; (FIG. 3G) Quantified inflammatory reaction volume and area measured by MRI and histology, respectively, showing that nBMP-2 caused less inflammation reaction than BMP-2. ** p<0.01, *** p<0.001.

FIG. 4. (FIG. 4A) Size distribution of native BSA and nBSA determined by dynamic light scattering (DLS). The average size of BSA is 6 nm, PDI=0.341. The average size of nBSA is 22 nm, PDI=0.656. (FIG. 4B) Surface zeta potential distribution of native BSA and nBSA. The average zeta potentials of BSA and nBSA are −20 mV and 8.4 mV, respectively.

FIG. 5. The degradation of nBSA(GDMA) with APm as cationic monomer. nBSA is incubated at 37° C. under different pH for 6 days.

FIG. 6. Photomicrographs of C3H10T1/2 cells treated with BMP-2 and nBMP2 after incubation in pH 8.5 buffer for different times.

FIG. 7. Schematic of making nanocapsules with sustained release capability. The synthesis was achieved through in situ polymerization of N-(3-aminopropyl) methacrylamide (APm, positively charged monomer), acrylamide (AAm, neutral monomer), and glycerol dimethacrylate (GDMA, degradable crosslinker) around the growth factors. Controlled degradation of GDMA under a basic pH environment breaks the shells and enables sustained release of the encapsulated proteins.

FIG. 8. nBMP-2 maintains longer time in therapeutic window than native BMP-2. The limit of the therapeutic window is an estimation, because it is really hard to be determined in the complicated biological system. Technically, the curve of BMP-2 release from nBMP-2 is not a typical sustained release curve, because it is an overall result of (1) BMP-2 release from the nanocapsules, (2) denaturation of free BMP-2 and (3) denaturation of BMP-2 in nanocapsules. As BMP-2 in the nanocapsules cannot be detected by ELISA, the apparent AUC is lower than that of free BMP-2.

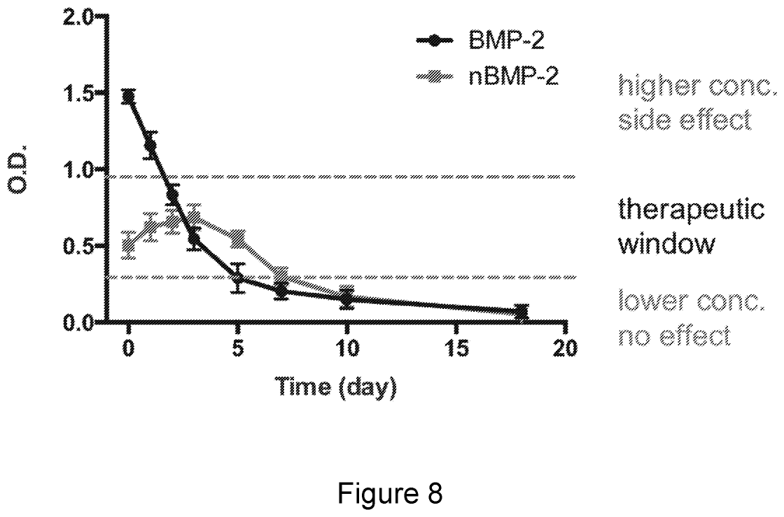

### DETAILED DESCRIPTION OF THE INVENTION

In the description of the preferred embodiment, reference may be made to the accompanying drawings which form a part hereof, and in which is shown by way of illustration a specific embodiment in which the invention may be practiced. It is to be understood that other embodiments may be utilized and structural changes may be made without departing from the scope of the present invention.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

All publications, patents, and patent applications cited herein are hereby incorporated by reference in their entirety for all purposes, including Lu et al. (PCT/US2010/026678), which describes a single-protein encapsulation technology and Wen et al., “Controlled Protein Delivery Based on Enzyme-Responsive Nanocapsules”, Advanced Materials, 2011, 23(39): 4549-4553, doi: 10.1002/adma.201101771, which describes a system for growth factor delivery aimed towards the control of angiogenesis.

As described above, growth factors and other proteins are generally unstable and short-lived in vivo. Delivery of proteins in vivo is further complicated by a desire for sustained release over a period of time rather than a burst or rapid release of the proteins. Current methods and strategies in the art for sustained release of proteins rely on reactions or chemical processes that may alter or compromise the activity of the proteins. The invention provides a novel protein delivery platform based on in-situ polymerization on individual protein molecules. The polymer forms a protective layer or shell around the internal proteins and can be degraded to release the protein cargos [24, 25]. Experimental data (disclosed in the Examples section below) have demonstrated that the protein cargo retain their bioactivity when released from the nanocapsule. Significantly, the degradation rate of the polymer shells can be controlled such that there is sustained release of the protein cargo.

Growth factors have a limited half-life and narrow therapeutic window. Coupled with wound healing, a very lengthy biological process, there is a clear need for a technology that can control and sustain the release of a growth factor locally. For example, bone regeneration requires the application of a highly precise level of bone morphogenetic protein-2 (BMP-2) to meet a narrow therapeutic window. As shown in experiments provided in the Example section, delivery of BMP-2 using this method resulted in a regenerated bone of better quality as well as with less inflammation when compared to the direct application of BMP-2 that is currently used clinically, therefore demonstrating a more advanced method for the administration of BMP-2 for the purposes of bone regeneration. As compared to current methods and strategies, the technology described herein can precisely control the release kinetics of growth factors administered in vivo. This unique feature is essential for the efficient and safe use of growth factors for many therapeutic purposes.

The invention disclosed herein has a number of embodiments. In one embodiment of the invention, a composition of matter is described that includes a degradable nanocapsule comprising a protein encapsulated within a polymer shell. The shell stabilizes the protein and can be degraded to release the protein [20, 21]. The degradable nanocapsule is formed by incorporating a degradable crosslinker during polymerization. This design enables extracellular release, for example in a bone regeneration environment by using a crosslinker that is degradable under alkaline conditions (e.g. a glycerol dimethacrylate (GDMA) crosslinker). In another example, an acid-labile protein nanocapsule is provided that releases the protein cargo in the acidic environment in endosomes [24]. Specifically, by incorporating acid-degradable crosslinkers, protein nanocapsules uptaken by cells release the protein cargo intracellularly, upon degradation of the shells within the acidic endosomes [20]. In a further example, by incorporating peptide-based crosslinkers in the shells, the crosslinkers may be degraded by specific enzymes to release the protein cargo [26, 27]. Based on this platform, a working embodiment of the invention provides growth-factor nanocapsules with sustained extracellular release capability for bone regeneration by using an alkaline-degradable crosslinker.

The kinetics of degradation and, thus, biologic drug release, are controlled not only by the selected degradable crosslinkers but also by further altering the polymer composition of the polymer shell. Specifically, the degradation rate of the polymer shell can be tuned by changing the ratio of the one or more different monomers forming the polymer shell (see, e.g. Table 1 in the Example section). In typical embodiments, the monomer is positively charged or neutral, and the crosslinker is an alkaline-degradable crosslinker. Examples of monomers that may be used to encapsulate the protein cargo (e.g. a growth factor such as a bone morphogenic protein) include various combinations and ratios of N-(3-aminopropyl) methacrylamide (APm), acrylamide (AAm), and 2-(dimethylamino)ethyl methacrylate (DMA). In one instance, the polymer shell comprises both N-(3-aminopropyl) methacrylamide (APm) and acrylamide (AAm). In another instance, the polymer shell comprises both N-(3-aminopropyl) methacrylamide (APm) and 2-(dimethylamino)ethyl methacrylate (DMA). Degradation of the polymer shell depends on the ratio of N-(3-aminopropyl) methacrylamide (APm) to acrylamide (AAm) or 2-(dimethylamino)ethyl methacrylate (DMA). In the example of BMP-2, nanocapsules with a slow release rate are the most suitable and thus do not include DMA monomer. However, in other applications, faster release may be desired and would include DMA monomer.

Although the protein cargo are typically encapsulated in polymer shells individually, the cleavage of the crosslinkers (e.g. ester bonds) does not happen simultaneously. The kinetics of bond cleavage thus allows for a gradual release of the protein cargo over a couple of days. For example, ratios of polymerized monomers and/or crosslinkers used to form a polymer nanocapsule may be controlled so that the polymeric nanocapsule does not degrade at a non-alkaline pH such as pH 7 but degrades at an alkaline pH such as pH 7.4 and above. Additionally, at a pH above 7.4, there is sustained release of the protein cargo. For instance, the population of encapsulated proteins can be designed so that the time required to release 50% of the protein cargo from a polymer nanocapsule is greater than 1, 2, 3, 4, 5, 10, 15 or 18 days.

Typically, the composition of matter is provided as a population of polymer nanocapsules. Each polymer nanocapsule comprises a protein cargo and a degradable polymer shell encapsulating the protein cargo. An illustrative embodiment of the invention is a composition comprising a population of polymer nanocapsules, with each of the polymer nanocapsules comprising a protein cargo and a polymer shell that encapsulates the protein cargo and which is degradable in alkaline environments such as in vivo sites of bone healing and repair. In such embodiments, the polymer shell is formed from alkaline-degradable crosslinkers and/or monomers, and individual polymer nanocapsules in the population of polymer nanocapsules are formed to have different amounts of crosslinkers and/or different monomers, thereby providing a variable and sustained release of the protein cargo from the population of nanocapsules in an environment having a pH of 7.4 or above. These populations of nanocapsules that provide a variable and sustained release of the protein cargo in alkaline environments can be formed from a number of constituents known in the art, for example glycerol dimethacrylate (GDMA) crosslinkers, and one or more different monomers are selected from the group consisting of N-(3-aminopropyl) methacrylamide (APm), acrylamide (AAm), and 2-(dimethylamino)ethyl methacrylate (DMA). In some embodiments of the invention, the polymer shell comprises both N-(3-aminopropyl) methacrylamide (APm) and acrylamide (AAm). Optionally the polymer shell comprises both N-(3-aminopropyl) methacrylamide (APm) and 2-(dimethylamino)ethyl methacrylate (DMA).

Typically, amounts each constituent used to form the nanocapsules is controlled so that 50% of the protein cargo from the population of polymer nanocapsules is released into the environment over a period of more than 1, 2, 3, 4, 5, 10 or 18 days. In some embodiments of the invention, less than 25% of the protein cargo from the population of polymer nanocapsules is released over a period of 6 days. In certain embodiments, the rate at which a polymer shell degrades in the environment is dependent on ratios of N-(3-aminopropyl) methacrylamide (APm) and the 2-(dimethylamino)ethyl methacrylate (DMA) used to form the polymer shells.

A wide variety of protein cargos can be used in embodiments of the invention. Typically, the protein cargo is a growth factor. In an exemplary embodiment, the growth factor is bone morphogenetic protein-2 (BMP-2), the monomer is selected from the group consisting of N-(3-aminopropyl) methacrylamide (APm), acrylamide (AAm), and 2-(dimethylamino)ethyl methacrylate (DMA); and/or the crosslinker is glycerol dimethacrylate (GDMA). In certain embodiments of the invention, the polymer nanocapsules in the population of nanocapsules have a diameter of less than 60 nm, 40 nm or 20 nm.

Another embodiment of the invention is a method for producing polymer nanocapsules disclosed herein. Typically, this method comprises selecting a core cargo molecule for encapsulation, selecting a plurality of shell monomers and/or cross-linkers having moieties that degrade at a pH of 7.4 or above. In these embodiments, amounts of crosslinkers and/or monomers used to make the thin polymer shell can be varied so as to form a population of nanocapsules having varying amounts of crosslinkers and/or different amounts of monomers disposed therein. These methods include physically adsorbing a plurality of shell monomers and cross-linkers to said core cargo molecule, where this adsorbing is modulated by electrostatic forces between the monomers and the core cargo molecule. The method includes polymerizing the plurality of adsorbed shell monomers and cross-linkers around said core cargo molecule to provide degradable nanocapsules formed from a thin polymer shell. In such embodiments, the amounts of crosslinkers and/or monomers varied to form a population of nanocapsules that are designed to variably degrade in environments having a pH above 7.4, 7.5, 7.6, 7.7, 7.8 or 7.9. Optionally, the population of nanocapsules is formed in batches that are subsequently mixed together to provide a variable and sustained release of the protein cargo from the population of nanocapsules.

In typical embodiments of the invention, the population of polymer nanocapsules provides a variable and sustained release of the protein cargo from a population of nanocapsules in an environment having a pH of 7.4 or above (e.g. an in vivo environment undergoing bone healing or regeneration). In some embodiments of the invention, the polymer nanocapsules are formed so that 50% of the protein cargo from the population of polymer nanocapsules is released over a period of more than 1, 2, 3, or 18 days. Optionally, the polymer nanocapsules are formed so that the protein cargo from the population of polymer nanocapsules is released over a period of at least 5 days. In certain embodiments, the growth factor is a bone morphogenetic protein, the monomer is selected from the group consisting of N-(3-aminopropyl) methacrylamide (APm), acrylamide (AAm), and 2-(dimethylamino)ethyl methacrylate (DMA); and/or the crosslinker comprises glycerol dimethacrylate (GDMA).

Another embodiment of the invention includes methods for delivering a protein cargo to an in vivo site. The method comprises delivering a polymer nanocapsule to an in vivo site and degrading the polymer shell such that the protein cargo is released at the site. The polymer nanocapsule comprises a protein cargo and a degradable polymer shell encapsulating the protein cargo. The polymer shell comprises polymerized monomers and crosslinkers. Furthermore, the polymer shell does not alter the bioactivity of the protein cargo. Embodiments of the invention also include methods for forming a polymer nanocapsule. The method comprises incubating a protein cargo with monomers and degradable crosslinkers and initiating free-radical polymerization to form a degradable polymer shell around the protein cargo. The monomers and crosslinkers surround the protein cargo through electrostatic interaction and/or hydrogen-bonding.

An illustrative embodiment of the invention is a method for stimulating bone regeneration comprising delivering a polymer nanocapsule that encapsulates a bone stimulating growth factor to bone tissue. In this method, the polymer nanocapsule can comprise a growth factor that stimulates bone regeneration (e.g. bone morphogenetic protein-2 (BMP-2) growth factor) and is formed from a degradable polymer shell that encapsulates the protein cargo. In such embodiments, the polymer shell can comprise at least one of polymerized N-(3-aminopropyl) methacrylamide (APm) and acrylamide (AAm) monomers and/or glycerol dimethacrylate (GDMA) crosslinkers. Typically, the polymer shell degrades in environments having a pH above 7.4; and degrading the polymer shell results in the growth factor being released at the bone tissue environments having a pH above 7.4 so as to stimulate bone regeneration. Typically, the method for stimulating bone regeneration results in less inflammation and/or adipogenesis when compared to delivering BMP-2 to the bone tissue in the absence of the polymer nanocapsule.

As noted above, specific embodiments of the invention include compositions and methods for stimulating bone regeneration. An illustrative polymer nanocapsule comprises a bone morphogenetic protein-2 (BMP-2) growth factor and a degradable polymer shell encapsulating the BMP-2. Typically, the nanoscale alkaline-degradable protein nanocapsule is formed via in situ polymerization on the growth factor. The polymer nanocapsule is delivered to a bone tissue and the polymer shell is degraded such that the bone morphogenetic protein-2 (BMP-2) is released at the bone tissue and stimulates osteoinduction. A slow release rate is most suitable for such applications. In a working embodiment of the invention, the polymer shell comprises polymerized N-(3-aminopropyl) methacrylamide (APm) and acrylamide (AAm) monomers and glycerol dimethacrylate (GDMA) crosslinkers. The polymer shell does not alter the bioactivity of the BMP-2. The BMP-2 loaded nanocapsules further reduce the side effects associated with the excessive use of native BMP-2 in traditional bone regenerative therapies. The method for stimulating bone regeneration results in less inflammation and/or adipogenesis when compared to delivering BMP-2 to the bone tissue without the polymer nanocapsule. In specific instances, the BMP-2 loaded nanocapsules provide improved therapeutic outcomes in spinal cord fusion.

Further aspects and embodiments of the invention are disclosed in the following examples.

### EXAMPLES

Proof of principle was first demonstrated with bovine serum albumin (BSA) and then the optimized parameters were used for delivering a therapeutic protein, bone morphogenetic protein-2 (BMP-2). The experimental results showed that the BMP-2 nanocapsules provide a more controlled and sustained delivery of the peptide for 5 days when compared to the native peptide control. This controlled release was shown in vitro using ALP activity, an indicator of osteoinduction, in C3H10T1/2 cells. Most importantly, the nanocapsule BMP-2 treatment described shows better bone fusion with higher quality bone as well as a reduction in side-effects (e.g. inflammation and adipogenesis) compared to free peptide.

### Example 1: Bovine Serum Albumin Nanocapsules (nBSA)

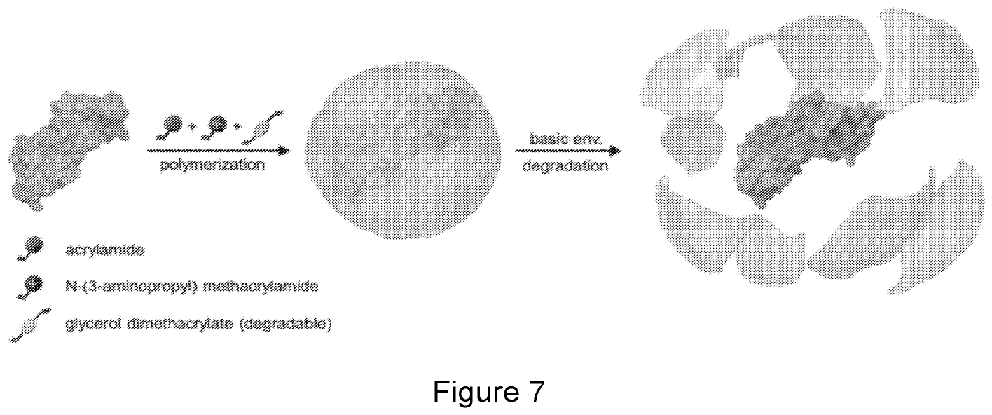

To demonstrate the synthesis of the nanocapsules with sustained release capability, bovine serum albumin (BSA) was first employed as a model protein. As illustrated in FIG. 7, the synthesis of the nanocapsules (denoted as nBSA) can be achieved by in-situ polymerization at 4° C. Briefly, BSA is firstly incubated with N-(3-aminopropyl) methacrylamide (APm, positively charged monomer), acrylamide (AAm, neutral monomer), and glycerol dimethacrylate (GDMA, degradable crosslinker). Electrostatic interaction and hydrogen-bonding enrich the monomers and crosslinkers around the protein. Free-radical polymerization is then initiated to form a thin layer of polymer network around the protein, leading to the formation of nBSA. In basic environment, the ester bonds in the crosslinker GDMA are gradually cleaved, leading to the dissociation of the polymer shells and the release of the protein cargo. The polymer shell composition can be readily altered to finely tune the degradation kinetics, allowing sustained release of the protein cargo with concentration maintained within a defined therapeutic window.

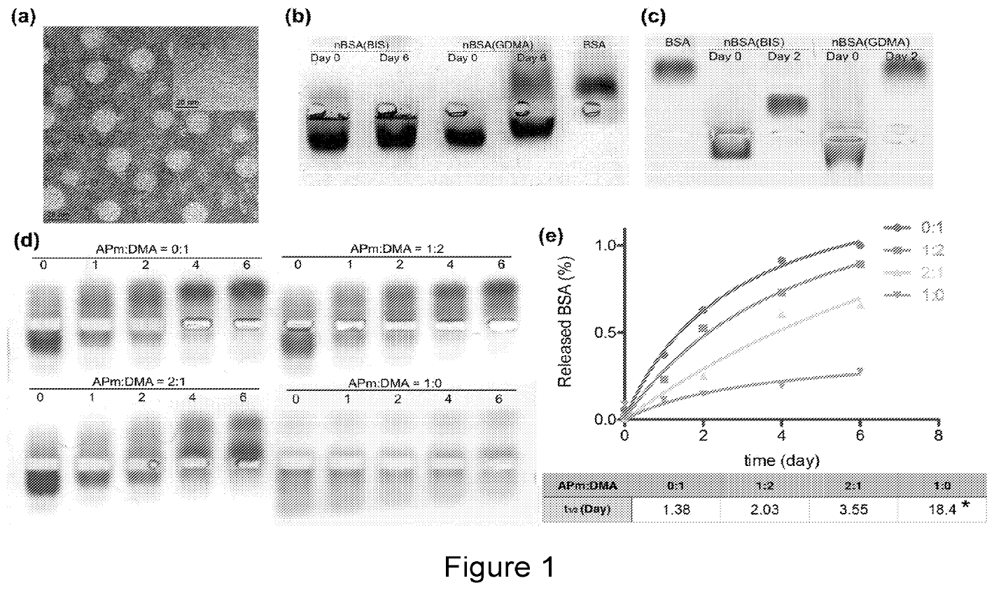

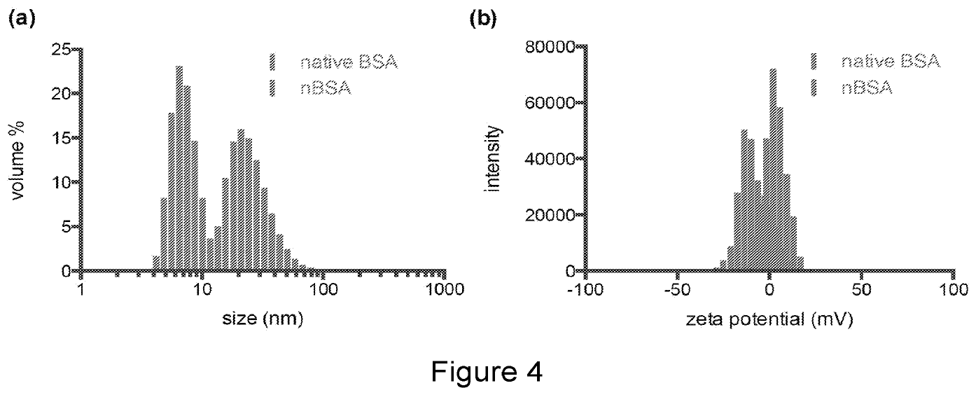

In a transmission electron microscopic (TEM) image (FIG. 1A) nBSA has spherical morphology with an average diameter about 20 nm. To better reveal the structure, similar nBSA was prepared with APm and N-[tris(hydroxymethyOmethyl]acrylamide as the monomer, allowing the polymer shell to be positively stained for TEM. As expected, the nanocapsules exhibit a core-shell structure (FIG. 1A, inset). In consistence with the TEM observation, dynamic light scattering (DLS) demonstrates that the mean diameter of the native BSA is −6 nm (FIG. 4A), whereas the diameter of nBSA reaches ˜22 nm. The mean ζ potential of the native BSA is around −20 mV. nBSA has a mean ζ potential of 8.4 mV (FIG. 4B), indicating the successful formation of the nanocapsules with cationic polymeric shells.

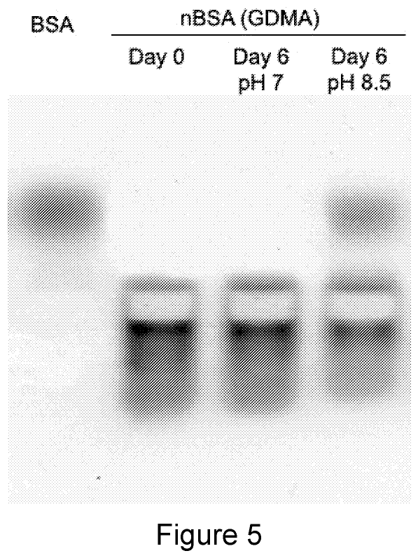

Previous studies indicate that bone repair is associated with a slightly decreased local pH value in the very early phase and later becomes more alkaline until the end of the healing process [37]. It was rationalized that alkaline-degradable nanocapsules would be ideal for BMP-2 delivery. Agarose gel electrophoresis was used to demonstrate the degradation of nBSA in an alkaline environment. As shown in FIG. 1B, native BSA migrates toward the anode under the electric field due to its negative surface charge. In contrast, the positively charged nBSA migrates to the cathode. After incubation in pH 8.5 for 6 days, degradable nBSA made with GDMA crosslinker (denote as nBSA(GDMA)) released the BSA cargo, which migrated to the similar position as that of the native BSA. In comparison, non-degradable nanocapsules (denoted as nBSA(BIS)), which were synthesized under similar condition by replacing the degradable GDMA crosslinker with bis-acrylamide (BIS, a non-degradable crosslinker), remains the same migrating behavior before and after alkaline exposure. A neutral environment, however, does not cause the degradation of nBSA (FIG. 5) during the 6-day incubation, indicating nBSA is reasonably stable at physiological environment.

To further tune the protein-release kinetics, degradable cationic monomers containing alkaline-labile ester bonds were also used, such as 2-(dimethylamino)ethyl methacrylate (DMA). As expected, nBSA made with DMA shows faster degradation kinetics than those made with APm. nBSA(GDMA) made with DMA and GDMA is mostly degraded within 2 days (FIG. 1C). It was found that nBSA(BIS) made with DMA and the non-degradable crosslinker BIS could not release the BSA cargo after incubation in the basic pH solution for 2 days. The surface charge of nBSA(BIS) is converted from positive to negative, which is due to the hydrolysis of DMA that created anionic carboxylate groups (FIG. 1C).

Given that the composition of the polymer shells can be readily controlled, this strategy enables fine tuning of the release kinetics simply by adjusting the ratio of APm and DMA used. FIG. 1D shows the agarose electrophoresis of nBSA(GDMA) made with different molar ratios of APm and DMA. During the 6-day incubation at pH 8.5, all four samples showed the release of BSA with rate decreasing with increasing APm/DMA ratio. Gel densitometry was used to quantify the release kinetics in FIG. 1D. The results (FIG. 1E) suggested that the half-release time (t1/2, time required to release 50% of the BSA cargo) increases from 1.38 days to 3.55 days when the APm molar percentage is increased from 0% to 33%. When the APm content is further increased to 100%, 22% cargo is released during the first 6 days. The adjustable release rate provides a platform for sustained release of growth factors (proteins) for various clinic applications. The studies presented above confirm the feasibility of making protein nanocapsules with tunable releasing capability in alkaline environment by adjusting the shell composition.

### Example 2: Bone Morphogenetic Protein-2 Nanocapsules (nBMP-2)

To translate this technology for BMP-2 mediated bone regeneration, a slow process that typically takes about 4-8 weeks, nanocapsule composition with slow release kinetics was chosen. In particular, BMP-2 nanocapsules (denoted as nBMP-2) were prepared with AAm and APm as the monomers and GDMA as the crosslinker.

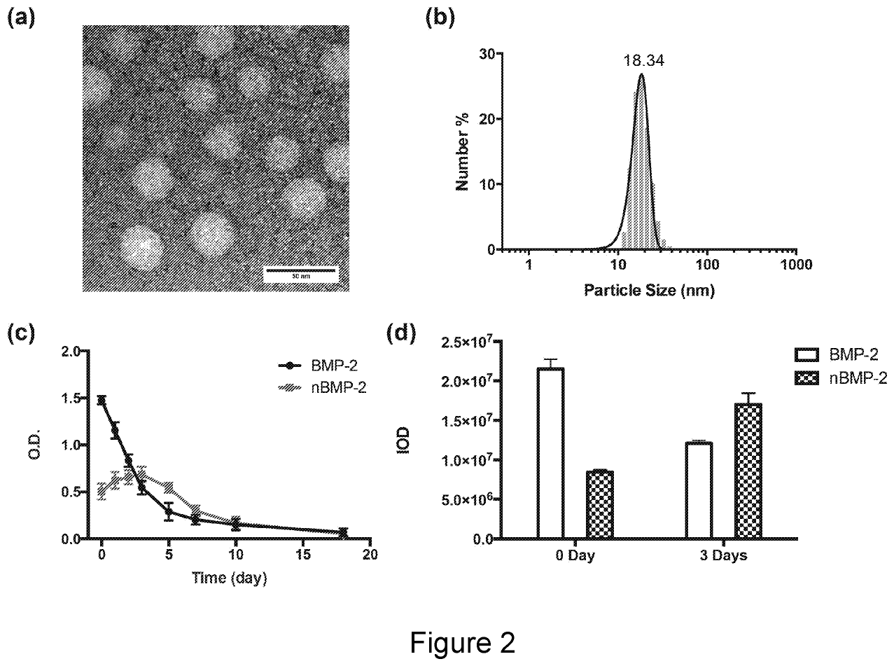

A TEM image of nBMP-2 (FIG. 2A) shows spherical morphology with an average diameter of around 20 nm, in consistence with the DLS measurement (FIG. 2B). FIG. 2C shows the release profile of BMP-2 (represented as optical density, OD) by enzyme-linked immunosorbent assay (ELISA) after incubating nBMP-2 in borate buffer (pH 8.5). For comparison, native BMP-2 with the same concentration was also incubated in borate buffer. The effective concentration of native BMP-2 declines significantly with incubation time, which is consistent with its poor stability. In contrast, effective BMP-2 concentration of the nBMP-2 sample remains at a comparatively stable level during the incubation. The initial OD for the nBMP-2 sample is around one third (⅓) of the native BMP-2 during the incubation. Assuming the BMP-2 retains the activity during the encapsulation at 4° C., it is estimated that around two third (2/3) of the BMP-2 were encapsulated within the nanocapsules (inaccessible to anti-BMP-2 antibodies). The nBMP-2 consistently releases BMP-2, resulting in increasing BMP-2 concentration with a maximum at day 3. The effective BMP-2 concentration decreases after the day 3, due to the activity decay of the released BMP-2 and the reducing concentration of nBMP-2. Overall, nBMP-2 provides comparatively stable BMP-2 concentration in alkaline environment. The sustained release system helps to maintain stable BMP-2 concentration for bone regeneration, avoiding undesired side effects caused by excessive amount of BMP-2.

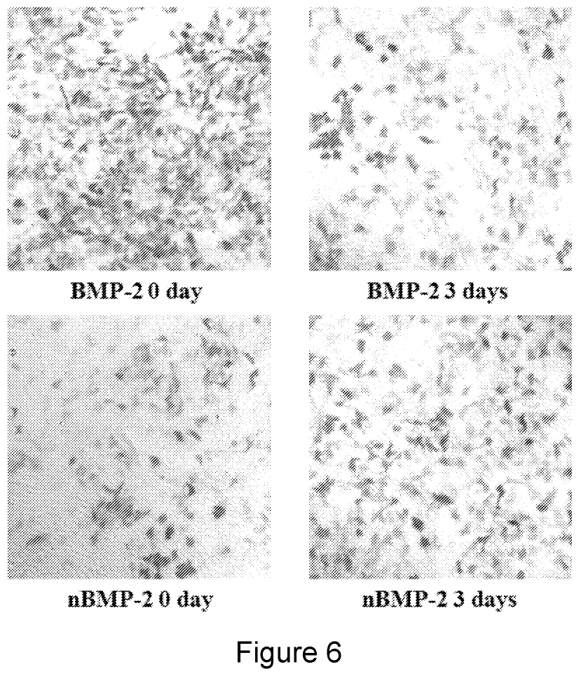

The controlled release of BMP-2 from the nanocapsules can stimulate osteoinduction in a sustained fashion. Osteogenic differentiation of murine mesenchymal stem cells C3H10T1/2 was used to assess the osteoinductive effect. During osteogenesis, the expression level of alkaline phosphatase (ALP) is up-regulated. The ALP activity was therefore chosen as an indicator for the osteoinductive effect. As shown in FIG. 6, the C3H10T1/2 cells exhibit deep purple color upon incubation with BMP-2 or nBMP-2, indicating the ability of both groups to stimulate bone regeneration. The ALP activity of C3H10T1/2 cells incubated with native BMP-2 was higher than the cells incubated with nBMP-2 on Day 0. Nevertheless, the ALP activity of the cells incubated with nBMP2 became higher on Day 3 (FIG. 2D). These observations indicate that although the native BMP-2 induces a stronger osteogenesis at the beginning of incubation, sustained release of BMP-2 from nBMP-2 would lead to prolonged osteogenesis.

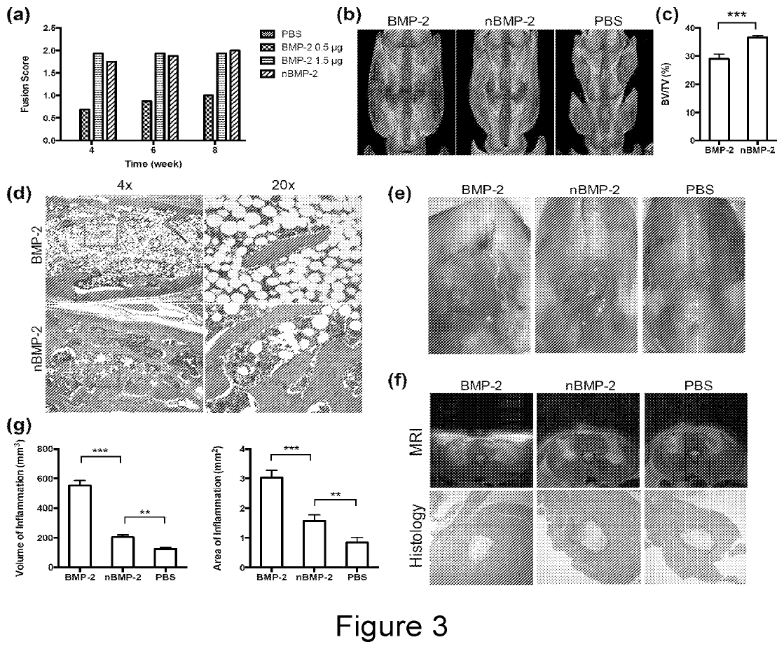

The posterolateral spinal fusion at L4-L5 in rat is a well-established animal model for spinal fusion. It is well accepted as an inexpensive and reliable in-vivo model to test the effects of bone grafting substitutes and enhancers on spinal fusion [38]. Similar to the FDA-approved use of recombinant human BMP-2, BMP-2 or nBMP-2 was implanted with absorbable collagen sponges in the intramuscular space of rats. At week 4, the spines of most rats in both the nBMP-2 group and the native BMP-2 group showed obvious bone growth and fusion on x-rays (FIG. 3A). The average fusion score of the nBMP-2 group was 1.75 at 4 weeks while that of the BMP-2 group was 1.94. Nevertheless, at week 8, the average fusion score of the nBMP-2 group increased to 2; while the average fusion score for the BMP-2 group stayed unchanged (1.94). The higher fusion score indicates a better bone quality of the regenerated tissue. Similar results were seen with the micro CT images at week 8 (FIG. 3B). The average relative bone volume (BV/TV) of the nBMP-2 group was 36.6±0.7% whereas the value for the BMP-2 group was 29.0±1.7%, suggesting that the quality of the new bone formed in the presence of nBMP-2 was higher (FIG. 3C). These observations collectively confirm the sustained bone regeneration mediated by nBMP-2.

Histological examination further reveals that the quality of the bone stimulated by nBMP-2 is better than that by native BMP-2. As shown in FIG. 3D, both BMP-2 and nBMP-2 groups demonstrate bridging bone at L4-L5 with clear evidence of trabecular and cortical bone forming the fusion masses, while the specimens from the PBS control group had no significant bone formation in the intertransverse process space. Significantly greater adipocyte formation within the fusion mass was seen in specimens from the BMP-2 group compared to those from the nBMP-2 group, which further substantiated the better quality of bone in the nBMP-2 group (FIG. 3d). It has been reported that BMP-2 overdosing dysregulates Wnt signaling and activates PPARy to promote adipogenesis over osteoblastogenesis, leading to inconsistent bone formation as well as decreased bone quality [30, 33, 39]. Although low doses of BMP-2 are desired to improve the bone quality, this would easily result in nonunion due to the short half-life of native BMP-2. The use of nBMP-2 enables sustained release of BMP-2 at an appropriate level, avoiding the adipogenesis without sacrificing bone regeneration or causing the nonunion effect.

Controlled release of BMP-2 from nBMP-2 also reduces the side effects caused by inflammation. Although inflammatory response is the initial step in the process of BMP-2 mediated bone regeneration, it also causes various side effects. In certain clinical applications such as cervical spine surgery, inflammatory edema caused by BMP-2 has resulted in swallowing/breathing difficulties or dramatic swelling, leading to paralysis or asphyxia in clinical applications. To address these side effects, emergency surgical evacuation would possibly be required [40-42]. To evaluate inflammation, soft-tissue edema volume was measured using a 7-Tesla magnetic resonance imaging (MRI) scanner 2 days post operation. Rats were euthanized after the MRI scans and sections were taken for histological tests. When dissecting the specimen, considerable amount of inflammatory edema overflew out of the incision, pervading the subcutaneous space (FIG. 3E). This is in accordance with the clinical setting, in which huge volume of edema would form after administration of BMP-2, causing serious complications. Inflammatory edema volume was quantified using MRI. Representative MR images from each group are shown in FIG. 3F and the mean inflammatory volume for each group is shown in FIG. 3G. On Day 2, the mean inflammatory volume of the BMP-2 group was significantly greater than those of the nBMP-2 and PBS groups (P<0.01). Histological studies yield similar conclusions. However, the inflammatory area surrounding the sponges from the nBMP-2 group is significantly smaller than those from the BMP-2 treated group, corroborating the MRI data. Overall, these results prove that the controlled release of BMP-2 effectively alleviates the inflammation response caused by high level of BMP-2. Due to the poor stability of native BMP-2, current bone regeneration treatment requires administrating an excessive amount of native BMP-2 to achieve complete union, inevitably leading to undesired inflammatory side effects. Therefore, the sustained release system of nBMP-2 nanocapsules provides a practical strategy for the safe and effective use of BMP-2 for bone regeneration.

To summarize, a nanoscale controlled release system has been established by encapsulating growth factors in polymeric nanocapsules. With BMP-2 mediated bone regeneration, an improved therapeutic outcome and mitigated side effects has been demonstrated. Compared to the direct use of native BMP-2, sustained release of BMP-2 from the nanocapsules successfully mediated bone regeneration, leading to bone regeneration with better bone quality. In addition, sustained release of BMP-2 reduces the side effects associated with the excessive use of native BMP-2 in the traditional spinal cord fusion surgery, providing a safe and more effective BMP-2 therapy for bone regeneration. Moreover, as a general method, this controlled release system may be extended for other therapeutic proteins in a variety of clinical applications.

### Example 3: Materials

All chemicals were purchased from Sigma-Aldrich unless otherwise noted, and were used as received. Cal-Ex decalcifying solution was purchased from Fisher Scientific (Fairlawn, N.J.). N-(3-Aminopropyl) methacrylamide was purchased from PolySciences, Inc (Warrington, Pa.). All cells were obtained from ATCC (Manassas, Va.). Cell culture dishes were purchased from Fisher Scientific (Pittsburgh, Pa.). All cell culture medium was purchased from Invitrogen (Grand Island, N.Y.). BMP-2 protein was obtained from Medtronic (Minneapolis, Minn.). BMP-2 ELISA Kit was purchased from R&D Systems, Inc (MN, USA). Alkaline Phosphatase kit was purchased from Sigma-Aldrich. Helistat collagen sponge was purchased from Integra Life Sciences (Plainsboro, N.J.). All sutures were purchased from Ethicon Inc. (Somerville, N.J.). All animals were purchased from Charles River Laboratories (Hollister, Calif.).

### Example 4: Instruments

UV-Visible adsorption was acquired with a Beckman Coulter DU®730 UV/Vis Spectrophotometer. TEM images were obtained on a Philips EM-120 TEM instrument. Agarose gel electrophoresis was obtained with an Edvotek M6Plus Electrophoresis Apparatus. Fluorescence intensities and ELISA result were measured with a Fujifilm BAS-5000 plate reader. Videos were tapped with a Canon Legria FS 406 Digital Camcorder. Fourier Transformed Infrared Spectroscopy (FT-IR) was acquired with JASCO FT/IR-420 spectrometer. High-speed burr was purchased from Medtronic (Minneapolis, Minn.). X-ray was done by using a Cabinet X-ray System from Faxitron Bioptics, LLC (Tucson, Ariz.). Micro-computed tomography (micro-CT) was scanned using a SkyScan 1172 scanner (Kontich, Belgium). MRI scans were performed by using the Bruker 7-T MRI scanner (Bruker Biospin Co, Fremont, Calif.). Micro-CT Virtual image slices were reconstructed using the cone-beam reconstruction software version 2.6 based on the Feldkamp algorithm (SkyScan), Sample re-orientation and 2D visualization were performed using DataViewer (SkyScan), and 3D visualization was performed using Dolphin Imaging version 11 (Dolphin Imaging & Management Solutions, Chatsworth, Calif.). Quantification of MR images was performed using Medical Image Processing, Analysis & Visualization (MIPAV, Version 5.3.3, NIH, Bethesda, Md.) computer software.

### Example 5: Experimental Methods

The Preparation of nBSA for Structural Characterization.

To a glass vial containing 1 mg BSA (5 mg/mL) and 100 μL 100 mM pH 7.0 phosphate buffer, 10.6 μL acrylamide (AAm, 20%, m/v), 13.4 μL N-(3-aminopropyl) methacrylamide hydrochloride (APm, 20%, m/v), and 2.6 μL glycerol diamethacrylate (GDMA, 10% m/v) were added. Then an appropriate amount of DI-water was added to reach a final volume of 1 mL. The solution was thoroughly mixed. Free radical polymerization was initiated by adding 10.3 μl of ammonium persulfate (APS, 10%, m/v) and 2.7 μl of N,N,N′,N′-tetramethylethylenediamine (TEMED). The reaction was allowed to proceed for 2 hr at 4° C., and then was extensively dialyzed against 10 mM pH 7.0 phosphate buffer using a cellulose membrane (MWCO 10 kDa) to remove the unreacted monomers and initiators. The yielded nanocapsules were used fresh or stored at −80° C. for future use. The nanocapsules prepared with the above protocol were used for DLS measurement, zeta potential measurement and TEM imaging (negative staining).

The Preparation of nBSA for TEM Positive Staining.

To a glass vial containing 1 mg BSA (5 mg/mL) and 100 μL 100 mM pH 7.0 phosphate buffer, 26 μL N-[Tris(hydroxymethyl)methyl]acrylamide (Tris, 20%, m/v), 13.4 μL N-(3-aminopropyl) methacrylamide hydrochloride (APm, 20%, m/v), and 2.6 μL glycerol diamethacrylate (GDMA, 10% m/v) were added (see Table 1 for monomer/crosslinker amounts). Then an appropriate amount of DI-water was added to reach a final volume of 1 mL. The solution was thoroughly mixed. Free radical polymerization was initiated by adding 10.3 μl of ammonium persulfate (APS, 10%, m/v) and 2.7 μl of N,N,N′,N′-tetramethylethylenediamine (TEMED). The reaction was allowed to proceed for 2 hr at 4° C., and then was extensively dialyzed against 10 mM pH 7.0 phosphate buffer using a cellulose membrane (MWCO 10 kDa) to remove the unreacted monomers and initiators. The yielded nanocapsules were used fresh or stored at −80° C. for future use. The nanocapsule prepared by this protocol were used for TEM imaging (positive staining).

The Preparation of nBSA for Degradation Assays.

Before in-situ polymerization, 5 mL 10 mg/mL BSA was thoroughly dialyzed against 10 mM pH 9.0 carbonate buffer. Subsequently, 5.8 μL fluorescein isothiocyanate (1 mg/mL in DMSO) was added to the dialyzed the BSA during gentle stirring. After the reaction was carried out overnight at 4° C., the labeled BSA was dialyzed against 10 mM pH 7 phosphate buffer and then adjusted to a final concentration of 5 mg/mL. To a glass vial containing 1 mg FITC-BSA (5 mg/mL) and 100 μL 100 mM pH 7.0 phosphate buffer, defined amounts of acrylamide (AAm, 20%, m/v), N-(3-aminopropyl) methacrylamide hydrochloride (APm, 20%, m/v), N,N-Dimethylaminoethyl Methacrylate (DMA, 20%, m/v), and glycerol diamethacrylate (GDMA, 10% m/v) or N,N′-Methylenebisacrylamide (BIS, 10%, m/v) were added (see Table 1 for monomer/crosslinker amounts). Then an appropriate amount of DI-water was added to reach a final volume of 1 mL. The solution was thoroughly mixed. Free radical polymerization was initiated by adding 10.3 μl of ammonium persulfate (APS, 10%, m/v) and 2.7 μl of N,N,N′,N′-tetramethylethylenediamine (TEMED). The reaction was allowed to proceed for 2 hr at 4° C., and then was extensively dialyzed against 10 mM pH 7.0 phosphate buffer using a cellulose membrane (MWCO 10 kDa) to remove the unreacted monomers and initiators. The yielded nanocapsules were used fresh or stored at −80° C. for future use. The nanocapsules prepared with this protocol were used for degradation assays.

TABLE 1
The recipe for nBSA preparation with different APm/DMA ratio (unit = μL)

| | BSA | AAm | APm | DMA | GDMA | APS | TEMED | Buffer | H₂O |
|---|---|---|---|---|---|---|---|---|---|
| conc. | 5 mg/mL | 20% w/v | 20% w/v | 20% w/v | 10% w/v | 10% w/v | — | 100 mM | — |
| APm:DMA = 0:1 | 200 | 10.6 | 0 | 11.8 | 2.6 | 10.3 | 2.7 | 100 | 662 |
| APm:DMA = 1:2 | 200 | 10.6 | 4.5 | 7.9 | 2.6 | 10.3 | 2.7 | 100 | 661 |
| APm:DMA = 2:1 | 200 | 10.6 | 8.9 | 3.9 | 2.6 | 10.3 | 2.7 | 100 | 661 |
| APm:DMA = 1:0 | 200 | 10.6 | 13.4 | 0 | 2.6 | 10.3 | 2.7 | 100 | 660 |

### Dynamic Light Scattering (DLS) Measurement.

DLS experiments were performed with a Zetasizer Nano instrument (Malvern Instruments Ltd., UK) equipped with a 10-mW helium-neon laser (2\, =632.8 nm) and thermoelectric temperature controller. Measurements were taken at 90° scattering angle. The samples are measured for size distribution and zeta potential distribution in a pH 7.0 10 mM phosphate buffer with a protein concentration of 1 mg/mL.

### Tem Measurement.

TEM images were obtained on a Philips EM-120 transmission electro microscopy. For negative stained nanocapsules, 10 μL 1 mg/mL nBSA or nBMP-2 was dropped on a copper grid. After 5 min, the solution was drawn off from the edge of the grid with filter paper. 5 μL of 1% pH=7.0 phosphotungstic acid (PTA) solution was immediately added on top of the grid. After another 5 min, the grid was washed 3 times with DI-water and allowed to dry in air. The grid was then stored for TEM observation. For positively stained nanocapsules, 10 μL of 1 mg/mL nBSA (with N-[Tris(hydroxymethyl)methyl]acrylamide and APm as monomers) and 10 μL of 2% pH=7.0 phosphotungstic acid (PTA) solution were mixed. After 1 hour, the mixture was dropped on a copper grid. After 2 min, the solution was drawn off from the edge of the grid with filter paper. The grid was then washed 3 times with DI water and allowed to dry in air. The grid was then stored for TEM observation. TEM images are acquired with an acceleration voltage of 120 kV and magnification of 67000× to 100000×

nBSA degradation assay under basic condition.

To 500 μL nBSA solutions, equal volumes of 100 mM pH 7.0 phosphate buffer or 100 mM pH 8.5 borate buffer were added and thoroughly mixed. The mixture was incubated at 37° C.; and at different time point, a 50-4 aliquot was transferred to a microcentrifuge tube to store at −80° C. After all the aliquots were collected, the degradation was visualized with an agarose gel electrophoresis. After the electrophoresis, the gel was imaged with a fluorescent gel imaging dock. Gel densitometry was used to quantify the releasing kinetics. acrylamide (AAm, 20%, m/v), 1.34 μL N-(3-aminopropyl) methacrylamide hydrochloride (APm, 20%, m/v) and 0.17 μL glycerol diamethacrylate (GDMA, 10% m/v) were added and thoroughly mixed in a 20 mM pH 6.0 MES buffer. Free radical polymerization was initiated by adding 0.34 μl of ammonium persulfate (APS, 10%, m/v) and 0.9 μL of N, N, N′, N′-tetramethylethylenediamine (TEMED, 10% m/v, adjusted to pH 6.0). The reaction was allowed to proceed for 2 hr at 4° C., and then was extensively dialyzed against 20 mM pH 7.0 phosphate buffer using a cellulose membrane (MWCO 10 kDa) to remove unreacted monomers and initiators. The yielded nanocapsules were used fresh or stored at −80° C. for future use. The nBMP2 prepared according to this protocol was used in further TEM, DLS, ELISA, cellular and in vivo studies.

Synthesis of nBMP-2.

To synthesize nBMP-2, 10 μL of BMP-2 (1.5 mg/mL), 0.53 μL acrylamide (AAm, 20%, m/v), 1.34 μL N-(3-aminopropyl) methacrylamide hydrochloride (APm, 20%, m/v) and 0.17 μL glycerol diamethacrylate (GDMA, 10% m/v) were added and thoroughly mixed in a 20 mM pH 6.0 MES buffer. Free radical polymerization was initiated by adding 0.34 μL of ammonium persulfate (APS, 10%, m/v) and 0.9 μL of N, N, N′, N′-tetramethylethylenediamine (TEMED, 10% m/v, adjusted to pH 6.0). The reaction was allowed to proceed for 2 hr at 4° C., and then was extensively dialyzed against 20 mM pH 7.0 phosphate buffer using a cellulose membrane (MWCO 10 kDa) to remove unreacted monomers and initiators. The yielded nanocapsules will be used fresh or stored at −80° C. for future use. The nBMP2 prepared according to this protocol was used in further TEM, DLS, ELISA, cellular and in vivo studies.

Release Kinetics of nBMP-2.

A BMP-2 ELISA Kit was purchased from R&D Systems, Inc (MN, USA). After encapsulation, borate buffer (100 mM pH=8.5) with 0.2 mg/ml BSA was added to both nBMP-2 and BMP-2 (final concentration: molar equivalent to 1.5 ug/mL BMP-2) to reach a basic condition. Both nBMP-2 and BMP-2 were then incubated at 37° C. During the incubation, samples were taken from both groups at Day 0, Day 1, Day 2, Day 3, Day 5, Day 7, Day 10, and Day 18 respectively, and then kept in −80° C. freezer. After collecting all samples, ELISA tests were carried out according to the manufactures' instructions. The plate was read at 450 nm with a correction wavelength of 540 nm. O.D. values were calculated into concentrations according to the standard curve generated with the standard BMP-2 samples.

Osteoinductive effect of nBMP-2 protein.

C3H10T1/2 cells were obtained from ATCC and maintained with 5% CO2 at 37° C. in Dulbecco's modified Eagle's medium (DMEM) supplemented with 10% fetal bovine serum (FBS) and 1% penicillin/streptomycin. Cells were plated in 24 well plates at 2×104 cells/ml and cultured for 24 h to allow cell attachment. After incubation, the culture medium was replaced with reduced serum medium (1% FBS) and incubated for another 12 h. After the incubation, cells were rinsed and cultured with 10% FBS DMEM, 10 uL of native BMP-2 (BMP-2, Day 0), freshly prepared nBMP-2 (nBMP2, Day 0), native BMP-2 incubated at 37° C. for 3 days (BMP-2, Day 3), and nBMP-2 incubated at 37° C. for 3 days (nBMP-2, Day 3) were added into the C3H10T1/2 cells and incubated for 96 h, respectively. After the incubation, cells were rinsed and cultured with 10% FBS DMEM for another 4 days. Cells were then stained using an alkaline phosphatase staining kit (Sigma-Aldrich), and the resulting images were analyzed using Image Pro software for the quantification of the alkaline phosphatase activities.

### Rat Spinal Fusion Surgery.

Twenty-four rats were allocated to 3 different groups according to different materials added to the implants. Group 1: 1.5 μg nBMP-2; Group 2: 1.5 μg native BMP-2; Group 3: PBS (control). Animals were anesthetized with 2% isoflurane administered in oxygen (1 L/min) and the surgical site was shaved and disinfected with alternative betadine and 70% ethanol. Animals were premedicated with 0.15 mg buprenorphine and after surgery received tapered doses every 12 hours for 2 days. The iliac crest was used as a landmark to locate the body of the L6 vertebra. A 4-cm longitudinal midline incision was made through the skin and subcutaneous tissue over L4-L5 down to the lumbodorsal fascia. Then 2-cm longitudinal paramedial incisions were made in the paraspinal muscles bilaterally. The transverse processes of L4-L5 were exposed, cleaned of soft tissue, and decorticated with a high-speed burr (Medtronic, Minneapolis, Minn.). The surgical site was then irrigated with sterile saline, and 5×5×12 mm pieces of collagen sponge (Helistat, Integra Life Sciences, Plainsboro, N.J.) containing 20 μl nBMP-2, BMP-2 or PBS were placed bilaterally, taking care to apply the implant to fully cover the transverse processes. The paraspinal muscles were then allowed to cover the implants, and the lumbodorsal fascia and skin were then closed. Animals were allowed to ambulate, eat, and drink ad libitum immediately after surgery.

### Radiological Examination of Spinal Fusion Result.

Posteroanterior radiographs were taken on each animal at 4 and 8 weeks post-surgery by using a cabinet X-ray system (Faxitron Bioptics, LLC, Tucson, Ariz.). Radiographs were evaluated blindly by 3 independent spine surgeons employing the following standardized scale: 0: no fusion; 1: incomplete fusion with bone formation present; and 2: complete fusion [43]. After 8 weeks follow up, the rats were euthanized by CO2 inhalation, and the lumbar spine specimens were then harvested. The explanted spines were subsequently scanned using high resolution micro-computed tomography (micro-CT), using a SkyScan 1172 scanner (SkyScan, Belgium) with a voxel isotropic resolution of 20 microns and an x-ray energy of 55 kVp and 167 mA to further assess the fusion rate and observe the fusion mass. 3D visualization was performed using Dolphin Imaging version 11 (Dolphin Imaging & Management Solutions, Chatsworth, Calif.). Fusion was defined as the bilateral presence of bridging bone between the L4 and L5 transverse processes. The reconstructed images were judged to be fused or not fused by 3 experienced independent observers.

### Histological Examinations of Rat Fusion Specimens.

After CT scan, the specimens were decalcified using a commercial decalcifying solution (Cal-Ex, Fisher Scientific, Fairlawn, N.J.), washed with running tap water, then transferred to 75% ethanol. The specimens were imbedded in paraffin and sagittal sections were cut carefully at the level of the transverse process to expose transverse process plane. These sections were stained with hematoxylin and eosin for histological imaging. Histologic sections were evaluated by an experienced independent observer.

### Surgical Procedure of the Rat Soft-Tissue Inflammation Model.

Eighteen rats were allocated to 3 different groups based on the samples absorbed by the ACS. Group 1: 20 μg nBMP-2; Group 2: 20 μg BMP-2; Group 3: PBS. Surgeries were done using our previous reported technique [44, 45]. Briefly, all animals were anesthetized with isoflurane inhalation and skins were sterilized with isopropyl alcohol and povidone-iodine. A 3-cm longitudinal midline incision was made through the skin and subcutaneous tissue over L3-L5 down to the lumbodorsal fascia. Then 2-cm longitudinal paramedial incisions were made in the paraspinal muscles bilaterally, using a longitudinal muscle splitting approach for intramuscular implantation of the sponge into the paraspinal muscle. The incision was made 10 mm from the midline along the lumbar spine, and the depth and length of the incision were kept below 10 mm. ACS (15 mm×5 mm×5 mm) with different samples were placed at the level of the L3-L5 spinous processes. The fascia and skin incisions were then closed.

### Quantified MRI Measurement of the Inflammatory Area.

Soft-tissue edema volume was measured as an index of inflammation after sponge implantation using a 7-Tesla small-animal MRI scanner (Bruker 7-T MRI scanner, Bruker Biospin Co, Fremont, Calif.). MRI scans were performed on Day 2, since according to the previous study, the mean inflammatory volume increases to a peak in all groups on Day 2, and equalizes between groups on Day 7. Day 0 MRI scans were saved because of the previous finding showing no difference between groups on Day 0 [45]. Axial sequences with a slice thickness of 1 mm were imaged. The volume of soft tissue edema was quantified from these MR images by two experienced independent observers, using Medical Image Processing, Analysis & Visualization software (MIPAV, Version 5.3.3, NIH, Bethesda, Md.).

### Histological Evaluation of the Inflammatory Area.

Rats were scarified after receiving the last MRI Scan. Soft tissue including muscle and the implants were excised and fixed in 10% formalin for histological analysis of the intramuscular implants. Specimens were dehydrated and embedded in paraffin. The length of the specimen, which included the length of the sponge, was 1 cm. Four cross-sections, each 0.25 mm thick, were taken through the sponge and surrounding muscle was stained with hematoxylin and eosin. The slides were analyzed by employing a quantitative scoring method to measure the area of the inflammatory zone surrounding the implant using ImageScope viewing software (Aperio, ImageScope Viewer) and MIPAV. The mean of the two sections with maximum dimension were used to calculate the inflammatory area for each animal.

### Abbreviations

BSA, bovine serum albumin; BMP-2, bone morphogenetic protein 2; APm, N-(3-aminopropyl) methacrylamide; AAm, Acrylamide; BIS, Bisacrylamide; GDMA, glycerol dimethacrylate; DMA, 2-(dimethylamino)ethyl methacrylate; TEM, transmission electron microscope; DLS, dynamic light scattering; OD, optical density; ELISA, enzyme-linked immunosorbent assay; ALP, alkaline phosphatase; MRI, magnetic resonance imaging.

### REFERENCES

Note: This application references a number of different publications as indicated throughout the specification by reference numbers enclosed in brackets, e.g., [x]. A list of these different publications ordered according to these reference numbers can be found below.

All publications mentioned herein are incorporated herein by reference to disclose and describe the methods and/or materials in connection with which the publications are cited. Publications cited herein are cited for their disclosure prior to the filing date of the present application. Nothing here is to be construed as an admission that the inventors are not entitled to antedate the publications by virtue of an earlier priority date or prior date of invention. Further, the actual publication dates may be different from those shown and require independent verification.
[1] Werner, S.; Grose, R. Regulation of Wound Healing by Growth Factors and Cytokines. Physiol. Rev. 2003, 83, 835-870.
[2] Martino, M. M.; Briquez, P. S.; Maruyama, K.; Hubbell, J. A. Extracellular Matrix-inspired Growth Factor Delivery Systems for Bone Regeneration. Adv. Drug Deliv. Rev.
[3] Bishop, G. B.; Einhom, T. A. Current and Future Clinical Applications of Bone Morphogenetic Proteins in Orthopaedic Trauma Surgery. Int. Orthop. 2007, 31, 721-727.
[4] Leader, B.; Baca, Q. J.; Golan, D. E. Protein Therapeutics: a Summary and Pharmacological Classification. Nat. Rev. Drug Discov. 2008, 7, 21-39.
[5] Tabata, Y. The Importance of Drug Delivery Systems in Tissue Engineering. Pharm. Sci. Technol. Today 2000, 3, 80-89.
[6] Vlodaysky, I.; Bar-Shavit, R.; Ishai-Michaeli, R.; Bashkin, P.; Fuks, Z. Extracellular Sequestration and Release of Fibroblast Growth Factor: a Regulatory Mechanism? Trends Biochem. Sci. 1991, 16, 268-271.
[7] Wachiralarpphaithoon, C.; Iwasaki, Y.; Akiyoshi, K. Enzyme-degradable Phosphorylcholine Porous Hydrogels Cross-linked with Polyphosphoesters for Cell Matrices. Biomaterials 2007, 28, 984-993.
[8] Ennett, A. B.; Kaigler, D.; Mooney, D. J. Temporally Regulated Delivery of VEGF in Vitro and in Vivo. J. Biomed. Mater. Res. A 2006, 79A, 176-184.
[9] Kanematsu, A.; Yamamoto, S.; Ozeki, M.; Noguchi, T.; Kanatani, I.; Ogawa, O.; Tabata, Y. Collagenous Matrices as Release Carriers of Exogenous Growth Factors. Biomaterials 2004, 25, 4513-4520.
[10] Lee, K. Y.; Peters, M. C.; Mooney, D. J. Controlled Drug Delivery from Polymers by Mechanical Signals. Adv. Mater. 2001, 13, 837-839.
[11] Hiemstra, C.; Zhong, Z.; van Steenbergen, M. J.; Hennink, W. E.; Feijen, J. Release of Model Proteins and Basic Fibroblast Growth Factor from in Situ Forming Degradable Dextran Hydrogels. J. Controlled Release 2007, 122, 71-78.
[12] Tessmar, J. K.; Gopferich, A. M. Matrices and Scaffolds for Protein Delivery in Tissue Engineering. Adv. Drug Deliv. Rev. 2007, 59, 274-291.
[13] Lee, H. C.; Kim, S.-J.; Kim, K.-S.; Shin, H.-C.; Yoon, J.-W. Remission in Models of Type 1 Diabetes by Gene Therapy Using a Single-chain Insulin Analogue. Nature 2000, 408, 483-488.
[14] Ulijn, R. V.; Bibi, N.; Jayawarna, V.; Thornton, P. D.; Todd, S. J.; Mart, R. J.;
Smith, A. M.; Gough, J. E. Bioresponsive Hydrogels. Mater. Today 2007, 10, 40-48.
[15] Mann, B. K.; Schmedlen, R. H.; West, J. L. Tethered-TGF-β Increases Extracellular Matrix Production of Vascular Smooth Muscle Cells. Biomaterials 2001, 22, 439-444.
[16] Hsu, B. B.; Jamieson, K. S.; Hagerman, S. R.; Holler, E.; Ljubimova, J. Y.; Hammond, P. T. Ordered and Kinetically Discrete Sequential Protein Release from Biodegradable Thin Films. Angew. Chem. Int. Ed. 2014, 53, 8093-8098.
[17] Alberto, F.; Bignon, C.; Sulzenbacher, G.; Henrissat, B.; Czjzek, M. The Three-dimensional Structure of Invertase ({beta}-Fructosidase) from Thermotoga Maritima Reveals a Bimodular Arrangement and an Evolutionary Relationship Between Retaining and Inverting Glycosidases. J. Biol. Chem. 2004, 279, 18903.
[18] Richardson, T. P.; Peters, M. C.; Ennett, A. B.; Mooney, D. J. Polymeric System for Dual Growth Factor Delivery. Nat. Biotechnol. 2001, 19, 1029-1034.
[19] Wang, Y.; Cooke, M. J.; Sachewsky, N.; Morshead, C. M.; Shoichet, M. S. Bioengineered Sequential Growth Factor Delivery Stimulates Brain Tissue Regeneration after Stroke. J. Controlled Release 2013, 172, 1-11.
[20] Silva, A. K. A.; Richard, C.; Bessodes, M.; Scherman, D.; Merten, O.-W. Growth Factor Delivery Approaches in Hydrogels. Biomacromolecules 2009, 10, 9-18.
[21] Fischbach, C.; Mooney, D. J. Polymers for Pro- and Anti-angiogenic Therapy. Biomaterials 2007, 28, 2069-2076.
[22] Biondi, M.; Ungaro, F.; Quaglia, F.; Netti, P. A. Controlled Drug Delivery in Tissue Engineering. Adv. Drug Deliv. Rev. 2008, 60, 229-242.
[23] Lavik, E.; Langer, R. Tissue Engineering: Current State and Perspectives. Appl. Microbiol. Biotechnol. 2004, 65, 1-8.
[24] Yan, M.; Du, J.; Gu, Z.; Liang, M.; Hu, Y.; Zhang, W.; Priceman, S.; Wu, L.; Zhou, Z. H.; Liu, Z.; et al. A Novel Intracellular Protein Delivery Platform Based on Single-protein Nanocapsules. Nat. Nanotechnol. 2009, 5, 48-53.
[25] Liu, Y.; Du, J.; Yan, M.; Lau, M. Y.; Hu, J.; Han, H.; Yang, O. O.; Liang, S.; Wei, W.; Wang, H.; et al. Biomimetic Enzyme Nanocomplexes and Their Use as Antidotes and Preventive Measures for Alcohol Intoxication. Nat. Nanotechnol. 2013, 8, 187-192.
[26] Wen, J.; Anderson, S. M.; Du, J.; Yan, M.; Wang, J.; Shen, M.; Lu, Y.; Segura, T. Controlled Protein Delivery Based on Enzyme-Responsive Nanocapsules. Adv. Mater. 2011, 23, 4549-4553.
[27] Zhu, S.; Nih, L.; Carmichael, S. T.; Lu, Y.; Segura, T. Enzyme-Responsive Delivery of Multiple Proteins with Spatiotemporal Control. Adv. Mater. 2015, 27, 3620-3625.
[28] Valdes, M. A.; Thakur, N. A.; Namdari, S.; Ciombor, D. M.; Palumbo, M. Recombinant Bone Morphogenic Protein-2 in Orthopaedic Surgery: a Review. Arch. Orthop. Trauma Surg. 2009, 129, 1651-1657.
[29] Axelrad, T. W.; Einhorn, T. A. Bone Morphogenetic Proteins in Orthopaedic Surgery. Cytokine Growth Factor Rev. 2009, 20, 481-488.
[30] Zara, J. N.; Siu, R. K.; Zhang, X.; Shen, J.; Ngo, R.; Lee, M.; Li, W.; Chiang, M.; Chung, J.; Kwak, J.; et al. High Doses of Bone Morphogenetic Protein 2 Induce Structurally Abnormal Bone and Inflammation In Vivo. Tissue Eng. Part A 2011, 17, 1389-1399.
[31] Cunningham, N. S.; Paralkar, V.; Reddi, A. H. Osteogenin and Recombinant Bone Morphogenetic Protein 2B Are Chemotactic for Human Monocytes and Stimulate Transforming Growth Factor Beta 1 mRNA Expression. Proc. Natl. Acad. Sci. U S. A. 1992, 89, 11740-11744.
[32] Kawamura, M.; Urist, M. R. Human Fibrin Is a Physiologic Delivery System for Bone Morphogenetic Protein. Clin. Orthop. 1988, 235.
[33] Takada, I.; Kato, S. [Molecular mechanism of switching adipocyte/osteoblast differentiation through regulation of PPAR-gamma function]. Clin. Calcium 2008, 18, 656-661.
[34] Bae, S. E.; Choi, J.; Joung, Y. K.; Park, K.; Han, D. K. Controlled Release of Bone Morphogenetic Protein (BMP)-2 from Nanocomplex Incorporated on Hydroxyapatite-formed Titanium Surface. J. Controlled Release 2012, 160, 676-684.
[35] Suliman, S.; Xing, Z.; Wu, X.; Xue, Y.; Pedersen, T. O.; Sun, Y.; Doskeland, A. P.; Nickel, J.; Waag, T.; Lygre, H.; et al. Release and Bioactivity of Bone Morphogenetic Protein-2 Are Affected by Scaffold Binding Techniques in Vitro and in Vivo. J. Controlled Release 2015, 197, 148-157.
[36] Kim, T.-H.; Yun, Y.-P.; Park, Y.-E.; Lee, S.-H.; Yong, W.; Kundu, J.; Jung, J. W.; Shim, J.-H.; Cho, D.-W.; Kim, S. E.; et al. In Vitro and in Vivo Evaluation of Bone Formation Using Solid Freeform Fabrication-based Bone Morphogenic Protein-2 Releasing PCL/PLGA Scaffolds. Biomed. Mater. 2014, 9, 025008.
[37] Chakkalakal, D. A.; Mashoof, A. A.; Novak, J.; Strates, B. S.; McGuire, M. H. Mineralization and pH Relationships in Healing Skeletal Defects Grafted with Demineralized Bone Matrix. J. Biomed. Mater. Res. 1994, 28, 1439-1443.
[38] Salamon, M. L.; Althausen, P. L.; Gupta, M. C.; Laubach, J. The Effects of BMP-7 in a Rat Posterolateral Intertransverse Process Fusion Model. J. Spinal Disord. Tech. 2003, 16.
[39] Krause, U.; Harris, S.; Green, A.; Ylostalo, J.; Zeitouni, S.; Lee, N.; Gregory, C. A. Pharmaceutical Modulation of Canonical Wnt Signaling in Multipotent Stromal Cells for Improved Osteoinductive Therapy. Proc. Natl. Acad. Sci. 2010, 107, 4147-4152.
[40] Shields, L. B. E.; Raque, G. H.; Glassman, S. D.; Campbell, M.; Vitaz, T.; Harpring, J.; Shields, C. B. Adverse Effects Associated With High-Dose Recombinant Human Bone Morphogenetic Protein-2 Use in Anterior Cervical Spine Fusion. Spine 2006, 31.
[41] Smucker, J. D.; Rhee, J. M.; Singh, K.; Yoon, S. T.; Heller, J. G. Increased Swelling Complications Associated With Off-Label Usage of rhBMP-2 in the Anterior Cervical Spine. Spine 2006, 31.
[42] MacDonald, K. M.; Swanstrom, M. M.; McCarthy, J. J.; Nemeth, B. A.; Guliani, T. A.; Noonan, K. J. Exaggerated Inflammatory Response After Use of Recombinant Bone Morphogenetic Protein in Recurrent Unicameral Bone Cysts. J. Pediatr. Orthop. 2010, 30.
[43] Alanay, A.; Chen, C.; Lee, S.; Murray, S. S.; Brochmann, E. J.; Miyazaki, M.; Napoli, A.; Wang, J. C. The Adjunctive Effect of a Binding Peptide on Bone Morphogenetic Protein Enhanced Bone Healing in a Rodent Model of Spinal Fusion. Spine 2008, 33.
[44] Lee, K.-B.; Taghavi, C. E.; Song, K.-J.; Sintuu, C.; Yoo, J. H.; Keorochana, G.; Tzeng, S.-T.; Fei, Z.; Liao, J.-C.; Wang, J. C. Inflammatory Characteristics of rhBMP-2 In Vitro and in an In Vivo Rodent Model. Spine 2011, 36.
[45] Tan, Y.; Montgomery, S. R.; Aghdasi, B. G.; Inoue, H.; Kaner, T.; Tian, H.; Terrell, R.; Zhang, X.; Wang, J. C.; Daubs, M. D. The Effect of Corticosteroid Administration on Soft-Tissue Inflammation Associated With rhBMP-2 Use in a Rodent Model of Inflammation. Spine 2013, 38.

### CONCLUSION

This concludes the description of the preferred embodiment of the present invention. The foregoing description of one or more embodiments of the invention has been presented for the purposes of illustration and description. It is not intended to be exhaustive or to limit the invention to the precise form disclosed. Many modifications and variations are possible in light of the above teaching.

1. A composition comprising a population of polymer nanocapsules, said population of polymer nanocapsules each comprising:
a protein cargo; and
a degradable polymer shell encapsulating the protein cargo;
wherein:
the polymer shell is formed from alkaline-degradable crosslinkers and one or more different monomers; and
individual polymer nanocapsules within the population of polymer nanocapsules are formed to have different amounts of alkaline-degradable crosslinkers and/or different monomers, thereby providing a variable and sustained release of the protein cargo from the population of nanocapsules in an environment having a pH of 7.4 or above.

2. The composition of claim 1, wherein:
the crosslinker is glycerol dimethacrylate (GDMA); and
the one or more different monomers are selected from the group consisting of N-(3-aminopropyl) methacrylamide (APm), acrylamide (AAm), and 2-(dimethylamino)ethyl methacrylate (DMA).

3. The composition of claim 2, wherein 50% of the protein cargo from the population of polymer nanocapsules is released over a period of more than 1, 2, 3, 4, 5, 10 or 18 days.

4. The composition of claim 2, wherein less than 25% of the protein cargo from the population of polymer nanocapsules is released over a period of 6 days.

5. The composition of claim 1, wherein the protein cargo is a growth factor.

6. The composition of claim 5, wherein:
the growth factor is bone morphogenetic protein-2 (BMP-2);
the monomer is selected from the group consisting of N-(3-aminopropyl) methacrylamide (APm), acrylamide (AAm), and 2-(dimethylamino)ethyl methacrylate (DMA); and/or
the crosslinker is glycerol dimethacrylate (GDMA).

7. The composition of claim 6, wherein polymer shell comprises both N-(3-aminopropyl) methacrylamide (APm) and acrylamide (AAm).

8. The composition of claim 6, wherein polymer shell comprises both N-(3-aminopropyl) methacrylamide (APm) and 2-(dimethylamino)ethyl methacrylate (DMA).

9. The composition of claim 8, wherein rate at which a polymer shell degrades in the environment is dependent on ratios of N-(3-aminopropyl) methacrylamide (APm) and the 2-(dimethylamino)ethyl methacrylate (DMA) used to form the polymer shells.

10. The composition of claim 5, wherein the polymer nanocapsules in the population of nanocapsules have a diameter of less than 60 nm, 40 nm or 20 nm.

11. A method for producing polymer nanocapsules comprising:
(a) selecting a core cargo molecule for encapsulation;
(b) selecting a plurality of shell monomers and/or cross-linkers having moieties that degrade at a pH of 7.4 or above;
(c) physically adsorbing a plurality of shell monomers and cross-linkers to said core cargo molecule, wherein:
said adsorbing is modulated by electrostatic forces between the monomers and the core cargo molecule; and
varying amounts of crosslinkers and/or monomers used so as to form a population of nanocapsules having varying amounts of crosslinkers and/or different amounts of monomers disposed therein;
(d) polymerizing a polymeric shell comprising the plurality of adsorbed shell monomers and cross-linkers around said core cargo molecule to provide degradable nanocapsules;
wherein said nanocapsules are formed to degrade in environments having a pH above 7.4, 7.5, 7.6, 7, 7, 7.8 or 7.9.

12. The method of claim 11, wherein the population of polymer nanocapsules provides a variable and sustained release of the protein cargo from a population of nanocapsules in an environment having a pH of 7.4 or above.

13. The method of claim 11, wherein:
the growth factor is a bone morphogenetic protein;
the monomer is selected from the group consisting of N-(3-aminopropyl) methacrylamide (APm), acrylamide (AAm), and 2-(dimethylamino)ethyl methacrylate (DMA); and/or
the crosslinker comprises glycerol dimethacrylate (GDMA).

14. The method of claim 11, wherein the population of nanocapsules is formed in batches that are subsequently mixed together to provide a variable and sustained release of the protein cargo from the population of nanocapsules.

15. The method of claim 11, wherein the polymer nanocapsules are formed so that 50% of the protein cargo from the population of polymer nanocapsules is released over a period of more than 1, 2, 3, or 18 days.

16. The method of claim 11, wherein the polymer nanocapsules are formed so that the protein cargo from the population of polymer nanocapsules is released over a period of at least 5 days.

17. A method for stimulating bone regeneration comprising:
delivering a polymer nanocapsule to bone tissue, said polymer nanocapsule comprising:
a growth factor that stimulates bone regeneration; and
a degradable polymer shell encapsulating the growth factor;
wherein:
the polymer shell comprises at least one of polymerized N-(3-aminopropyl) methacrylamide (APm) and acrylamide (AAm) monomers and/or glycerol dimethacrylate (GDMA) crosslinkers;
the polymer shell does not alter the bioactivity of the growth factor; and
the polymer shell degrades in environments having a pH above 7.4; and
degrading the polymer shell such that the growth factor is released at the bone tissue so as to stimulate bone regeneration.

18. The method of claim 17, wherein the growth factor is a bone morphogenetic protein.

19. The method of claim 18, wherein the growth factor is bone morphogenetic protein-2 (BMP-2) growth factor.

20. The method of claim 19, wherein the method for stimulating bone regeneration results in less inflammation and/or adipogenesis when compared to delivering BMP-2 to the bone tissue in the absence of the polymer nanocapsule.
